(19) United States
(12) Patent Application Publication
(10) Pub. No.: US 2022/0146527 A1
(43) Pub. Date: May 12, 2022

(54) METHOD OF CREATING CHARACTERISTIC PROFILES OF MASS SPECTRA AND IDENTIFICATION MODEL FOR ANALYZING AND IDENTIFYING FEATURES OF MICROORGANISMS

(71) Applicants: Jang-Jih Lu, Taipei City (TW); Hsin-Yao Wang, Chiayi City (TW); Chia-Ru Chung, Taoyuan City (TW); Jorng-Tzong Horng, Taoyuan City (TW); Tzong-Yi Lee, Taoyuan City (TW)

(72) Inventors: Jang-Jih Lu, Taipei City (TW); Hsin-Yao Wang, Chiayi City (TW); Chia-Ru Chung, Taoyuan City (TW); Jorng-Tzong Horng, Taoyuan City (TW); Tzong-Yi Lee, Taoyuan City (TW)

(73) Assignee: Chang Gung University, Taoyuan City (TW)

(21) Appl. No.: 17/583,418

(22) Filed: Jan. 25, 2022

Related U.S. Application Data
(63) Continuation-in-part of application No. 16/833,811, filed on Mar. 30, 2020.

(30) Foreign Application Priority Data
Sep. 17, 2019 (TW) 108133321

(51) Int. Cl. G01N 33/68; G16B 40/10; H01J 49/00; H01J 49/40

(52) U.S. Cl. CPC G01N 33/6851; H01J 49/40; H01J 49/0036; G16B 40/10

(57) ABSTRACT

A method of creating characteristic profiles of mass spectra and identification model for analyzing and identifying microorganisms includes collecting m/z data of microorganisms having same features from MALDI-TOF MS; classifying the microorganisms; classifying the collected set of m/z data as a plurality of subsets; creating modified subsets by applying KDE to the subsets; creating first characteristic MS profiles based on the modified subsets; summarizing into a second characteristic MS profile; repeating above six steps to create second characteristic MS profiles; creating a training set comprising first matched vectors; training a machine learning system using the training set to establish a feature classification model; using MALDI-TOF MS to analyze microorganisms having unknown features; comparing m/z of MALDI-TOF MS spectrum of the microorganisms having unknown features with second characteristic MS profiles to obtain second matched vectors; using the feature classification model analyzing the second matched vectors; and identifying the microorganisms.

### CROSS-REFERENCE TO RELATED APPLICATION

The present application is a continuation in part of U.S. patent application Ser. No. 16/833,811, filed on Mar. 30, 2020, titled METHOD OF CREATING CHARACTERISTIC PROFILES OF MASS SPECTRA AND IDENTIFICATION MODEL FOR ANALYZING AND IDENTIFYING FEATURES OF MICROORGANIZMS. listing Lu, Jang-Jih, Wang, Hsin-Yao, Chung. Chia-Ru, Homg, Jorng-Tzong and Lee, Tzong-Yi as inventors. This application claims the priority benefit of Taiwan Patent application number 108133321 filed on Sep. 17, 2019.

### 1. FIELD OF THE INVENTION

The invention relates to a method of creating and analyzing mass spectrometer signals and more particularly to a method of creating characteristic profiles of mass spectra and identification model for analyzing and identifying features of microorganisms by analyzing mass spectrometry (MS) of their biomolecules. The characteristic profile is a protein expression pattern obtained by analyzing signals from matrix-assisted laser desorption ionization-time of flight mass spectrometry (MALDI-TOF MS) of isolated microorganisms of the same feature. The MALDI-TOF MS data of the isolated microorganisms are processed by density-based clustering to find a mass-to-charge ratio (m/z) with high probability of occurrence. The values of high probability of occurrence together form a characteristic profile for a specific feature of microorganisms. Then, machine learning methods are used to integrate the profiles from different features of microorganisms in order to create features classification models which are used to analyze matched vectors of the microorganisms having the unknown features, thereby identifying and analyzing the features of the microorganisms.

### 2. DESCRIPTION OF RELATED ART

Conventionally, technologies of using MS to identify the species of an unknown microorganism involve comparing the MS of the unknown isolated microorganism to those of known microorganisms in an isolated MS database, or comparing the isolated MS of the unknown microorganism to the characteristic MS species profiles of known microorganisms. In the approach of isolated MS database comparison, it is required to gather all the isolated MS data of known microorganisms in a database. However, microorganisms evolve constantly. Thus, it is required to gather a huge amount of MS data of known isolated microorganism in the database. Further, in the identification step, the comparison process of the isolated MS of the unknown microorganism in the large isolated MS database of known microorganisms is time consuming. A large data storage for efficient and accurate comparison is required. And in turn, complex hardware is required.

For solving the above problem, there is an intelligent method of creating characteristic profiles of mass spectra and identification model for analyzing and identifying features of microorganisms disclosed. The method can quickly process comparisons of mass spectrometer signals data. However, it is first required to discretize the data and then it uses density-based clustering to find an m/z with high probability of occurrence from the discretized data, thereby solving the problem of MS signals drifting in different batch tests. However, the discretization neither identifies the corresponding signals nor provides a possible drifting range. In short, it is not capable of identifying protein.

Thus, the need for improvement does exist.

### SUMMARY OF THE INVENTION

It is therefore one object of the invention to provide a method of creating characteristic profiles of mass spectra and identification model for analyzing and identifying microorganisms, comprising the steps of (1) collecting a set of mass-to-charge ratio (m/z) data of microorganisms having same features from a matrix-assisted laser desorption ionization-time of flight mass spectrometry (MALDI-TOF MS); (2) classifying the microorganisms having same features by species, sub-species, resistance to antibiotics, or toxicity; (3) classifying the collected set of m/z data as a plurality of subsets based on the classification of step (2); (4) creating a plurality of modified subsets by applying kernel density estimation to the subsets such that a plurality of characteristic peaks and ranges are defined; (5) creating a plurality of first characteristic MS profiles based on the characteristic peaks and ranges of the modified subsets; (6) summarizing the plurality of first characteristic MS profiles into a second characteristic MS profile; (7) repeating steps (1) to (6) to create the second characteristic MS profiles of a plurality of features of the microorganisms; (8) creating a training set comprising a plurality first matched vectors obtained by comparing m/z of MALDI-TOF MS spectrum of microorganism having known features with the second characteristic MS profiles; (9) training a machine learning system using the training set to establish a feature classification model; (10) using MALDI-TOF MS to analyze microorganisms having unknown features; (11) comparing the m/z of MALDI-TOF MS spectrum of the microorganisms having unknown features with the second characteristic MS profiles to obtain a plurality of second matched vectors; (12) using the feature classification model to analyze the second matched vectors; and (13) identifying the microorganisms having the unknown features.

Preferably, the machine learning system uses Support Vector Machine (SVM), Artificial Neuron Network (ANN), k Nearest Neighbor (kNN), Logistic Regression (LR), Fuzzy Logic, Bayesian Algorithms, Decision Tree Induction Algorithm (DT), Random Forest (RF), Deep Learning, or any combination thereof.

Preferably, the kernel density estimation are uniform kernel, triangular kernel, biweight kernel, triweight kernel, Epanechnikov kernel, or Gaussian kernel, or any combination thereof.

Preferably, the microorganisms are bacteria, molds, or viruses.

When the features of the microorganisms are species or subspecies, classifying the microorganisms is done by nucleic acid sequencing. When the feature of the microorganisms is resistance to antibiotics, classifying the microorganisms is done by disc diffusion, microdilution, macrodiluation, agar dilution, or E-test. When the feature of the microorganisms is toxicity, classifying the microorganisms is done by nucleic acid sequencing.

The method of the invention has the following advantages and benefits in comparison with the conventional art:

Precise m/z can be obtained: creating characteristic profiles of mass spectra and identification model for analyzing and identifying features of microorganisms facilitates the summarization of the m/zs of the characteristic peaks. It can solve the problem of MS signals being drifted or shifted in different batches of an experiment due to discretization and the problem of being incapable of correctly finding locations of the signals to be aligned. Therefore, corrected locations of the signals to be aligned can be found, precise m/z can be obtained, and identifying protein is made easy.

Both identification precision and resolution are greatly increased: creating characteristic profiles of mass spectra and identification model for analyzing and identifying features of microorganisms can greatly increase both identification precision and resolution. It can solve the problem of the conventional method of identifying microorganism species (e.g., Shigella and E. coli). Further, it can be easily extended to the identification of species, sub-species, resistance to antibiotics, or toxicity. With the increased precision of MS data analysis, healthcare employees can use the analysis result to correctly use antibiotics for infection control in near real time.

Signal drift or shift problem is solved. An m/z comparison of the invention can solve the signal drift problem in microorganism MALDI-TOF MS data when the MS data are acquired from different batches of an experiment. Creation of the matched vectors facilitates the construction of microorganism identification models using machine learning methods. Machine learning is characterized by high accuracy, high performance and high repeatability. Thus, the analysis results of MS signals of the invention can be widely used in many applications. And in turn, it decreases the requirement of manual operation and manual intervention. Finally, it improves greatly the reduction of both man power and cost.

The above and other objects, features and advantages of the invention will become apparent from the following detailed description taken with the accompanying drawings.

### BRIEF DESCRIPTION OF THE DRAWINGS

FIG. 1 is a flow chart of a method of creating characteristic profiles of mass spectra and identification model for analyzing and identifying features of microorganisms by analyzing the MS of their biomolecules according to the invention;

FIG. 2 is a diagram illustrating establishment of characteristic MS profiles;

FIG. 3 includes a first plot of a density versus m/z in the range of 4000 to 7000 for ST3 in which black blocks represent original m/z distributions and dashed lines represent kernel density estimation, a second plot of a density versus m/z in the range of 4000 to 7000 for ST42 in which black blocks represent original m/z distributions and dashed lines represent kernel density estimation, and a third plot of a density versus m/z in the range of 4000 to 7000 for other ST types in which black blocks represent original m/z distributions and dashed lines represent kernel density estimation according to the invention;

FIG. 4 is a table showing peak values and ranges of ST3, ST42 and other ST types;

FIG. 5 is a table of matched vectors versus ST3, ST42 and other ST types;

FIG. 6A includes a first plot of sensitivity versus 1-specificity for Random Forest (RF) in which a solid line represents a kernel density estimation and a dashed line represents density-based clustering according to the invention, a second plot of sensitivity versus 1-specificity for Support Vector Machine (SVM) in which a solid line represents a kernel density estimation and a dashed line represents density-based clustering according to the invention, and a third plot of sensitivity versus 1-specificity for Logistic Regression (LR) in which a solid line represents a kernel density estimation and a dashed line represents density-based clustering all in terms of ST3 according to the invention;

FIG. 6B includes a first plot of sensitivity versus 1—specificity for RF in which a solid line represents a kernel density estimation and a dashed line represents density-based clustering according to the invention, a second plot of sensitivity versus 1—specificity for SVM in which a solid line represents a kernel density estimation and a dashed line represents density-based clustering according to the invention, and a third plot of sensitivity versus 1—specificity for LR in which a solid line represents a kernel density estimation and a dashed line represents density-based clustering all in terms of ST42 according to the invention;

FIG. 6C includes a first plot of sensitivity versus 1—specificity for RF in which a solid line represents a kernel density estimation and a dashed line represents density-based clustering according to the invention, a second plot of sensitivity versus 1—specificity for SVM in which a solid line represents a kernel density estimation and a dashed line represents density-based clustering according to the invention, and a third plot of sensitivity versus 1—specificity for LR in which a solid line represents a kernel density estimation and a dashed line represents density-based clustering all in terms of other ST types according to the invention;

FIG. 7 is a table showing sensitivity of each of ST3, ST 42 and other ST types in terms of LR, RF and SVM versus kernel density estimation, density-based clustering, kernel density estimation, density-based clustering, kernel density estimation and density-based clustering; specificity of each of ST3, ST 42 and other ST types in terms of LR, RF and SVM versus kernel density estimation, density-based clustering, kernel density estimation, density-based clustering, kernel density estimation and density-based clustering; accuracy of each of ST3, ST 42 and other ST types in terms of LR, RF and SVM versus kernel density estimation, density-based clustering, kernel density estimation, density-based clustering, kernel density estimation and density-based clustering; and area under curve (AUC) of each of ST3, ST 42 and other ST types in terms of LR, RF and SVM versus kernel density estimation, density-based clustering, kernel density estimation, density-based clustering, kernel density estimation and density-based clustering according to the invention; and

FIG. 8 is a table showing accuracy in terms of machine learning method, LR, RF and SVM versus kernel density estimation and density-based clustering, and AUC in terms of machine learning method, LR, RF and SVM versus kernel density estimation and density-based clustering according to the invention; and

FIG. 9 schematically depicts taking resistance to antibiotics as an exemplary example.

### DETAILED DESCRIPTION OF THE INVENTION

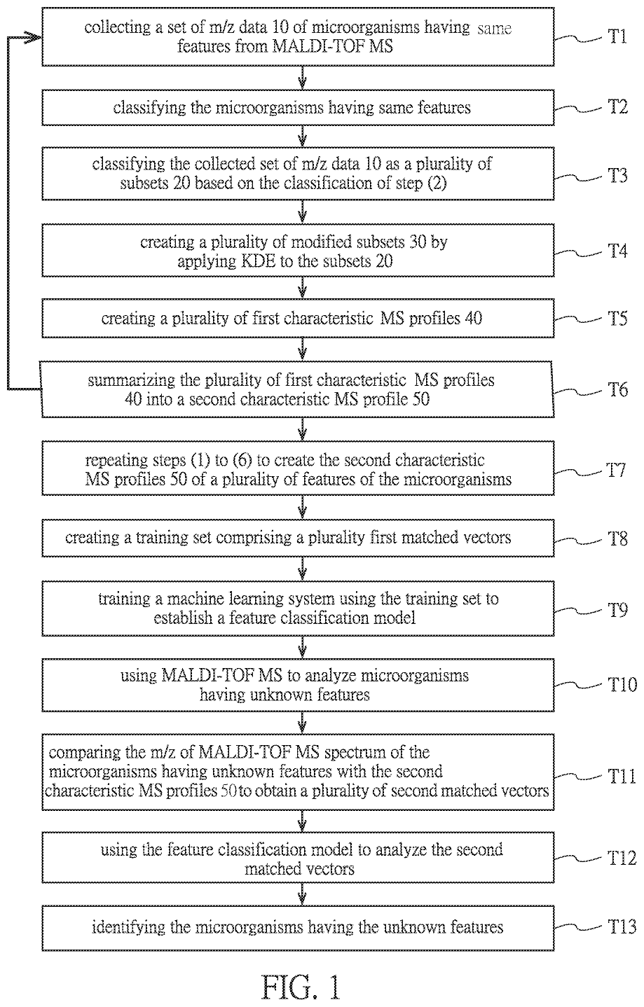

Referring to FIG. 1, a flow chart of a method of creating characteristic profiles of mass spectra and identification model for analyzing and identifying features of microorganisms by analyzing the MS of their biomolecules according to the invention is illustrated and comprises the steps of:

T1: collecting a set of mass-to-charge ratio (m/z) data 10 of microorganisms having same features from a matrix-assisted laser desorption ionization-time of flight mass spectrometry (MALDI-TOF MS);

T2: classifying the microorganisms having same features by species, sub-species, resistance to antibiotics, or toxicity;

T3: classifying the collected set of m/z data 10 as a plurality of subsets 20 based on the classification of step (2);

T4: creating a plurality of modified subsets 30 by applying kernel density estimation (KDE) to the subsets 20 such that a plurality of characteristic peaks and ranges are defined; wherein the kernel density estimation are uniform kernel, triangular kernel, biweight kernel, triweight kernel, Epanechnikov kernel, or Gaussian kernel;

T5: creating a plurality of first characteristic MS profiles 40 based on the characteristic peaks and ranges of the modified subsets 30;

T6: summarizing the plurality of first characteristic MS profiles 40 into a second characteristic MS profile 50;

T7: repeating steps (1) to (6) to create the second characteristic MS profiles 50 of a plurality of features of the microorganisms;

T8: creating a training set comprising a plurality first matched vectors obtained by comparing m/z of MALDI-TOF MS spectrum of microorganism having known features with the second characteristic MS profiles 50;

T9: training a machine learning system using the training set to establish a feature classification model;

T10: using MALDI-TOF MS to analyze microorganisms having unknown features;

T11: comparing the m/z of MALDI-TOF MS spectrum of the microorganisms having unknown features with the second characteristic MS profiles 50 to obtain a plurality of second matched vectors;

T12: using the feature classification model to analyze the second matched vectors; and

T13: identifying the microorganisms having the unknown features.

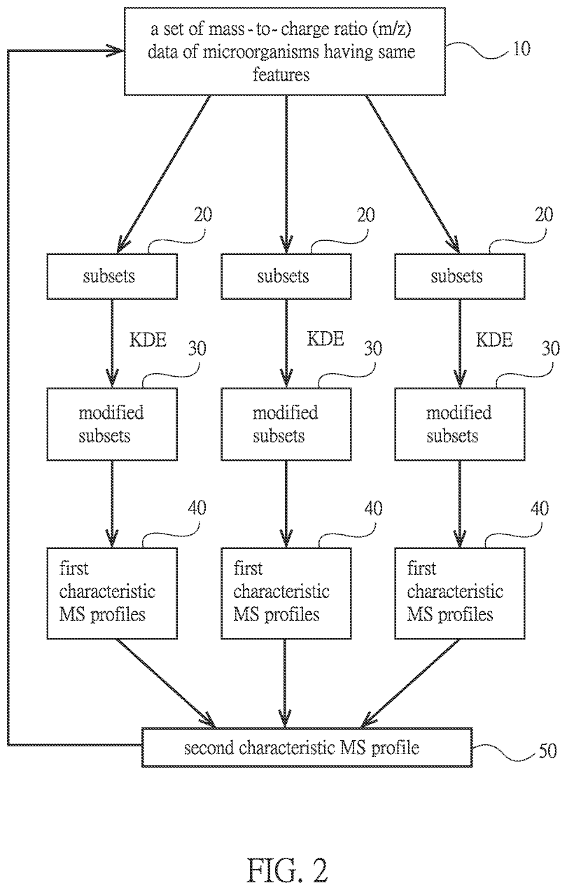

FIG. 2 illustrates establishment of characteristic MS profiles. A set of mass-to-charge ratio (m/z) data 10 of microorganisms having same features is classified as a plurality of subsets 20 by species, sub-species, resistance to antibiotics, or toxicity. Then, kernel density estimation (KDE) is applied to the subsets 20 to create a plurality of modified subsets 30 such that a plurality of characteristic peaks and ranges are defined. According to the characteristic peaks and ranges of each the modified subsets 30, a plurality of first characteristic MS profiles 40 are created. Next, the first characteristic MS profiles 40 are summarized into a second characteristic MS profile 50.

Sub-species of Staphylococcus haemolyticus is taken as an exemplary example in conjunction with FIG. 1 according to the invention in which MALDI-TOF MS collects data of 254 Staphylococcus haemolyticus. Next, Multi-Locus Sequence Typing (MLST) is used to identify sub-species of the Staphylococcus haemolyticus. The data include 15 sub-species in which ST3 and ST42 are of interest and data of other sub-species are few. Therefore, the data is classified as the subsets 20 of ST3, ST42 and other ST types. Then, kernel density estimation is applied to the subsets 20 of ST3, ST42 and other ST types to create the modified subsets 30.

Referring to FIG. 3, it includes a first plot of a density versus m/z in the range of 4000 to 7000 for ST3 in which black blocks represent original m/z distributions and dashed lines represent kernel density estimations, a second plot of a density versus m/z in the range of 4000 to 7000 for ST42 in which black blocks represent original m/z distributions and dashed lines represent kernel density estimation estimations, and a third plot of a density versus m/z in the range of 4000 to 7000 for other ST types in which black blocks represent original m/z distributions and dashed lines represent kernel density estimations according to the invention. In other words, the black blocks represent the subsets 20, and the dashed lines represent the modified subsets 30. In the modified subsets 30, characteristic peaks and ranges can be defined.

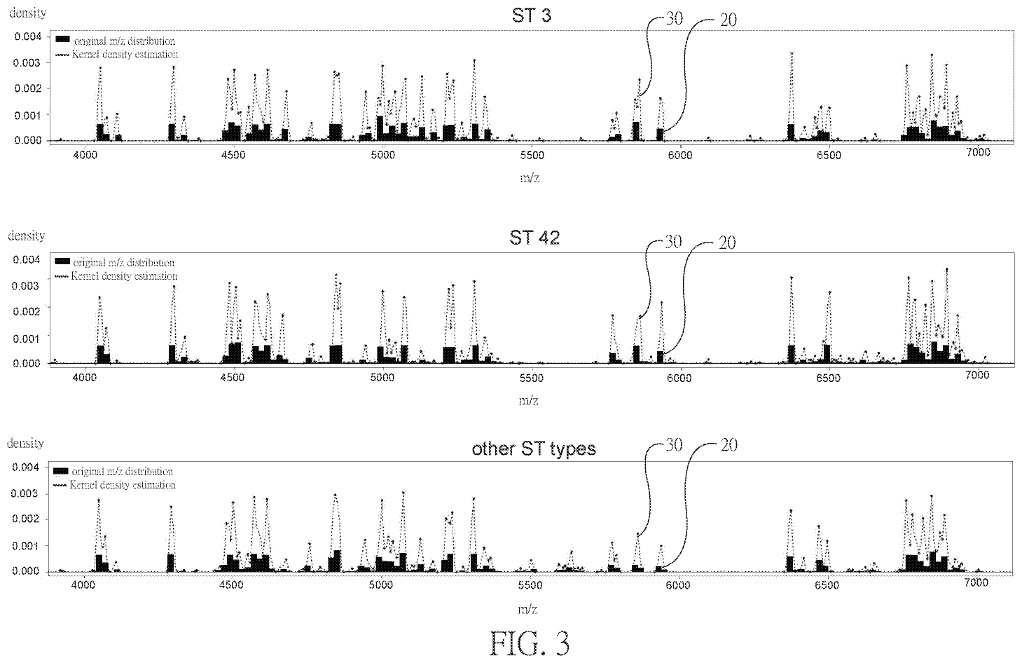

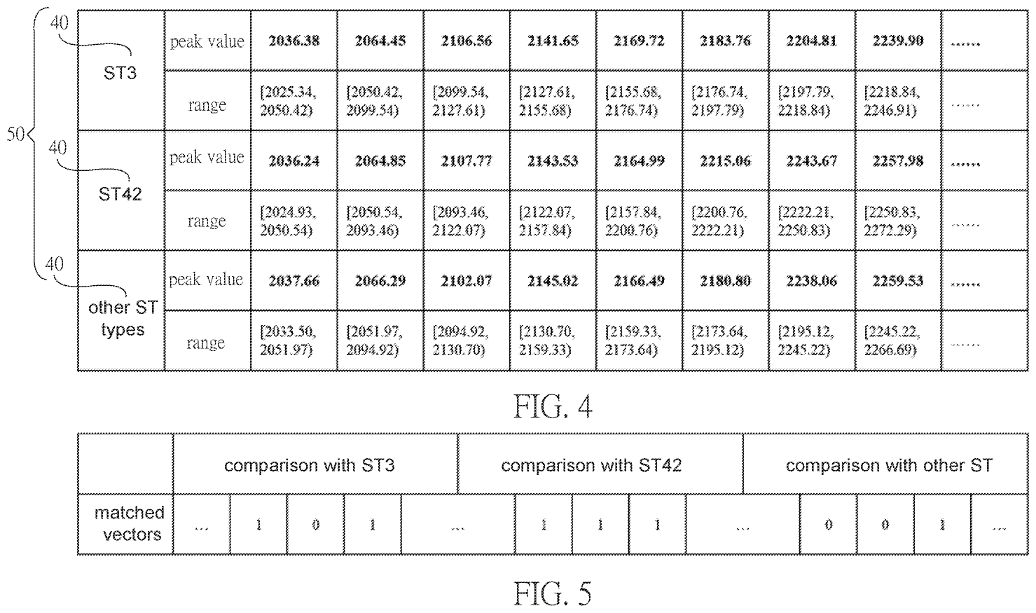

Referring to FIG. 4, it is a table showing characteristic peaks and ranges of ST3, ST42 and other ST types. To estimate the characteristic peaks, the distribution of m/z values to find out those possible true peaks' positions is important. Kernel density estimation can be applied in this situation to estimate the characteristic peaks. More X₁={x₁₁, x₁₂, . . . , x₁ₙ_{₁}}, X₂={x₂₁, x₂₂, . . . x₂ₙ_{₂}}, . . . , X_{N}={x_{N1}, x_{N2}, . . . , x_{Nn}_{ₓ}}, and then the equation: X={x₁₁, x₁₂, . . . , x₁ₙ_{₁}, x₂₁, x₂₂, . . . , x₂ₙ_{₁}, . . . , x_{N1}, . . . , x_{Nn}_{_{N}}} is formed. Since the distribution of X is not unimodal, use kernel density estimation to estimate a probability density function (PDF) of the respective ST types which can be written as the following equation:

$$f ( x ) = \frac{1}{h {\sum }_{k = 1}^{N} {n}_{k}} {\sum }_{i = 1}^{N} {\sum }_{j = 1}^{{n}_{i}} K ( \frac{x - {x}_{ij}}{h} )$$

where xij represents the m/z, h is the smoothing parameter, ni is the number of peaks in mass spectrum i, and K is the kernel function. The following Gaussian kernel function is applied:

$$K ( u ) = \frac{1}{\sqrt{2 π}} {e}^{- \frac{1}{2} {u}^{2}} .$$

After estimating the distribution, a localized mode is the characteristic peaks.

In FIGS. 3 and 4, signals distributions of different sub-species of microorganism are shown. A kernel density estimation is used to estimate m/z data of ST3, ST42 and other ST types respectively. Further, maximum and minimum area values are calculated and taken as aligned central points and drifting ranges. Finally, all characteristic peaks and its ranges are combined to obtain a model having aligned m/z.

As shown in FIG. 3, an MS signals distribution of each species of microorganism may be drifted. For example, molecules having an m/z of 4500 may generate a signal around 4500. However, a kernel density estimation may be used to process data not subjected to discretization to obtain a correct position of a characteristic peak.

As shown in FIG. 4, the second characteristic MS profile 50 of Staphylococcus haemolyticus is shown. The second characteristic MS profile 50 includes the first characteristic MS profiles 40 of ST3, ST42 and other ST types with characteristic peaks and ranges. For example, ST3 has a characteristic peak of 2036.38 and a covered range of 2025.34 to 2050.42. The m/zs represent the characteristic peaks of ST3. Location and possible drifting range of the m/z of each characteristic peak can be correctly defined based on the above information. A characteristic MS profile of a specific sub-species can be formed by summarizing the m/zs of the characteristic peaks.

Repeating the steps T1 to T6 until the second characteristic MS profiles 50 of a plurality of specific sub-species is obtained. After the second characteristic MS profiles 50 of the specific sub-species has been obtained, it is possible of comparing MS data of a plurality of known microorganisms sub-species with the second characteristic MS profile 50 of each sub-species in terms of signals to create a plurality of matched vectors as a training dataset. A plurality of different conventional machine learning methods are used to train the machine learning system and establish a sub-species classification identification model.

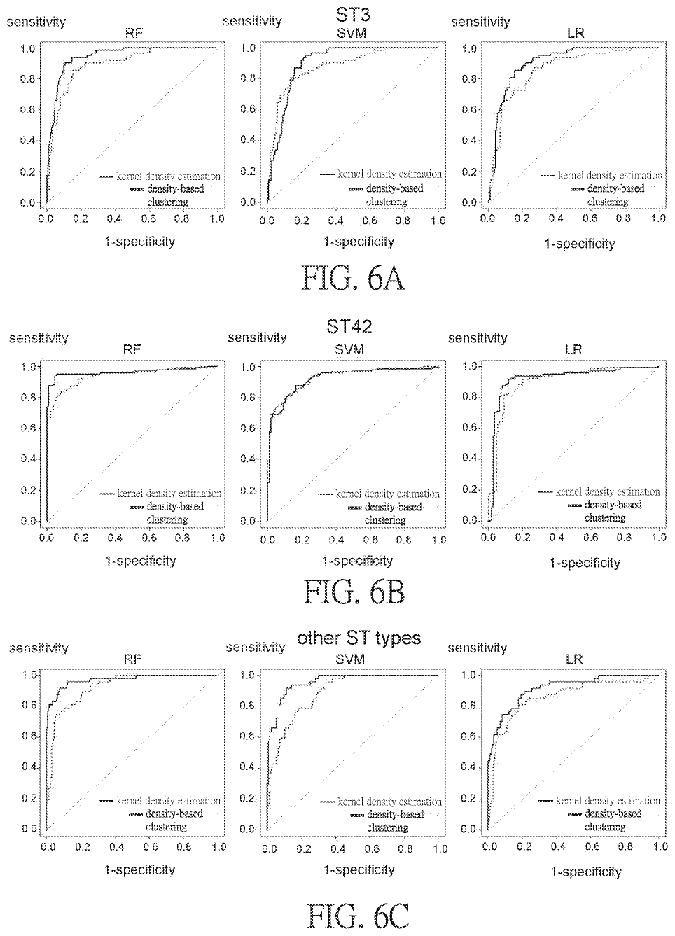

Referring to FIGS. 5 and 6, in an operation of unknown specimen, MALDI-TOF MS is used to obtain MS data of unknown microorganisms, and m/z data of each species is compared with the second characteristic MS profiles 50 in terms of signals to create a plurality of matched vectors which determine whether the MS signals of the unknown species are similar to that of each sub-species. As shown in FIG. 5, unknown microorganisms are compared with the second characteristic MS profile of each of ST3, ST42 and other ST types to obtain three different vectors which are labeled first, second and third vectors respectively based on the order of creating the matched vectors. Taking a comparison with the ST3 MS as an example, the first vector is 1, the second vector is 0, and the third vector is 1 in which 1 represents the existence of a signal peak in a specific m/z center and its covered range after the MS signals of the unknown microorganisms have compared with the ST3 MS; and to the contrary, 0 represents there is no signal peak of the m/z. After the three sub-species have been compared with the MS signals of the unknown microorganisms, the first, second and third vectors are concatenated to create a plurality of matched vectors of the unknown microorganisms. In fact, the matched vectors represent a characteristic of the unknown microorganisms and contain information of each species. The dimension of the vector is a fixed value in consideration of classification and identification so that a machine learning method can be used for analysis and determination.

Referring to FIGS. 6, 7 and 8 in which as shown in FIG. 6, the machine learning system uses three different machine learning methods are used in the embodiment including Logistic Regression (LR), Random Forest (RF) and Support Vector Machine (SVM); and kernel density estimation and density-based clustering are used respectively to create a dichotomy model of sub-species of each species. Its performance is excellent.

As shown in the dichotomy model of each of ST3, ST42 and other ST types of FIGS. 6A, 6B and 6C, a kernel density estimation is used to generate an MS of characteristic profiles. Irrespective of the machine learning method being used, an area under curve (AUC) of a receiver operating characteristic (ROC) curve is greater than 0.85 and density-based clustering is found. Further, the AUC of ROC curve is greater than 0.90 for an RF model in cooperation with kernel density estimation.

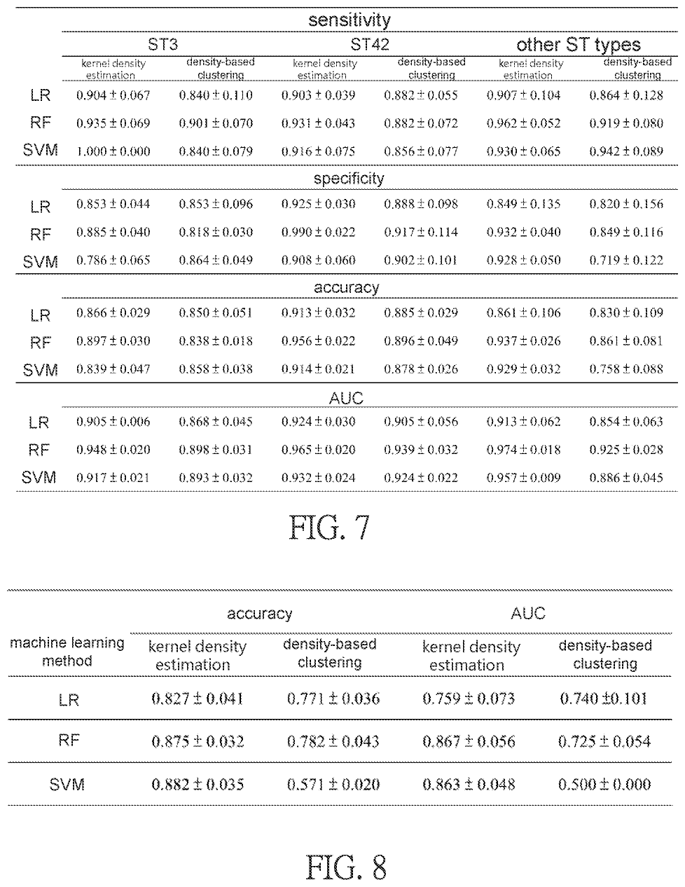

As shown in FIG. 7, it is found that there are many advantages of using kernel density estimation in each model. As shown in FIG. 8, a plurality of comprehensive classification identification models of sub-species are established in the embodiment. But being different from the dichotomy model, the comprehensive classification identification models of sub-species can do a plurality of times of classification and identification of sub-species in one time. In the embodiment, ST3, ST42 and other ST types can be identified in one time.

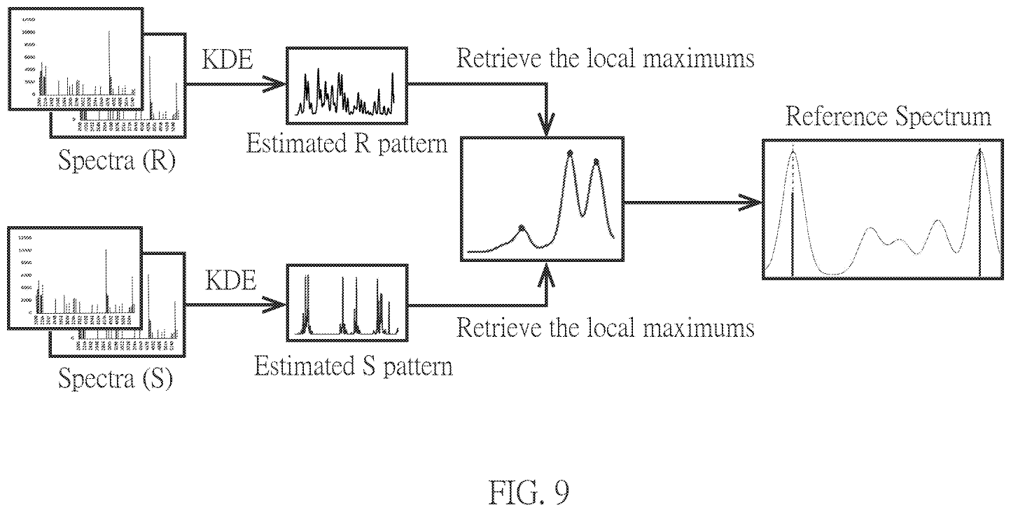

Referring to FIG. 9 and in conjunction with FIG. 1. Resistance to antibiotics is taken as another exemplary example as shown in FIG. 9. The microorganisms are classified as two subsets 20, resistant and susceptible to antibiotics. After applying kernel density estimation to the subsets 20 of resistant and susceptible to antibiotics respectively, the PDFs of m/z patterns for resistant and susceptible spectra were obtained. The local modes derived from two PDFs were retrieved and concatenated to be a one spectrum with several peaks. Then, the duplicate values to construct a reference spectrum template were removed. In addition to removing the duplicate values, the distance between two adjacent local modes less than three were also removed. Since the minimum width of two adjacent peaks expected in a spectrum was set as six m/z. Finally, these m/z values formed the final reference spectrum template.

In conclusion, kernel density estimation in cooperation with different machine learning methods can carry out an excellent identifying effect, e.g., having an accuracy of about 0.90 and being better than density-based clustering. Further, a standard deviation of the accuracy is very small and it means that the machine learning method has a very high accuracy.

It is clear from the above embodiment, the novel and nonobvious method of the invention can obtain more accurate characteristic MS profiles of species. Further, the machine learning methods being used can more precisely identify microorganism sub-species. It is understood that sub-species is a feature of microorganisms. In other words, the method of the invention can be easily extended to the identification of species, sub-species, resistance to antibiotics, or toxicity.

While the invention has been described in terms of preferred embodiments, those skilled in the art will recognize that the invention can be practiced with modifications within the spirit and scope of the appended claims.

What is claimed is:

1. A method of creating characteristic profiles of mass spectra and identification model for analyzing and identifying microorganisms, comprising the steps of:
(1) collecting a set of mass-to-charge ratio (m/z) data of microorganisms having same features from a matrix-assisted laser desorption ionization-time of flight mass spectrometry (MALDI-TOF MS);
(2) classifying the microorganisms having same features by species, sub-species, resistance to antibiotics, or toxicity;
(3) classifying the collected set of m/z data as a plurality of subsets based on the classification of step (2);
(4) creating a plurality of modified subsets by applying kernel density estimation to the subsets such that a plurality of characteristic peaks and ranges are defined;
(5) creating a plurality of first characteristic MS profiles based on the characteristic peaks and ranges of the modified subsets;
(6) summarizing the plurality of first characteristic MS profiles into a second characteristic MS profile;
(7) repeating steps (1) to (6) to create the second characteristic MS profiles of a plurality of features of the microorganisms;
(8) creating a training set comprising a plurality first matched vectors obtained by comparing m/z of MALDI-TOF MS spectrum of microorganism having known features with the second characteristic MS profiles;
(9) training a machine learning system using the training set to establish a feature classification model;
(10) using MALDI-TOF MS to analyze microorganisms having unknown features;
(11) comparing the m/z of MALDI-TOF MS spectrum of the microorganisms having unknown features with the second characteristic MS profiles to obtain a plurality of second matched vectors;
(12) using the feature classification model to analyze the second matched vectors; and
(13) identifying the microorganisms having the unknown features.

2. The method of claim 1, wherein the machine learning system uses Support Vector Machine (SVM), Artificial Neuron Network (ANN), k Nearest Neighbor (kNN), Logistic Regression (LR), Fuzzy Logic, Bayesian Algorithms, Decision Tree Induction Algorithm (DT), Random Forest (RF), Deep Learning, or any combination thereof.

3. The method of claim 1, wherein the microorganisms are bacteria, molds, or viruses.

4. The method of claim 1, wherein the kernel density estimation are uniform kernel, triangular kernel, biweight kernel, triweight kernel, Epanechnikov kernel, or Gaussian kernel, or any combination thereof.

5. The method of claim 1, wherein the features of the microorganisms are species or subspecies, classifying the microorganisms is done by nucleic acid sequencing.

6. The method of claim 1, wherein the feature of the microorganisms is resistance to antibiotics, classifying the microorganisms is done by disc diffusion, microdilution, microdilution, agar dilution, or E-test.

7. The method of claim 1, wherein the feature of the microorganisms is toxicity, classifying the microorganisms is done by nucleic acid sequencing.
